(19) United States
(12) Patent Application Publication
(10) Pub. No.: US 2025/0302898 A1
(43) Pub. Date: Oct. 2, 2025

(54) METHOD FOR TREATING MELANOMA WITH OHSV2

(71) Applicant: Binhui Biopharmaceutical Co., Ltd, Wuhan (CN)

(72) Inventor: Binlei LIU, Beijing (CN)

(21) Appl. No.: 18/623,168

(22) Filed: Apr. 1, 2024

(51) Int. Cl. A61K 35/763; A61K 41/00; A61P 35/00; C12N 7/00

(52) U.S. Cl. CPC A61K 35/763; A61K 41/0047; A61P 35/00; C12N 7/00; C12N 2710/16021; C12N 2710/16632

(57) ABSTRACT

Provided is an antitumor drug for melanoma, including recombinant oncolytic herpes simplex virus type II (oHSV2) in form of injection. The oHSV2 injection provided in the present disclosure is used for the treatment of subjects with melanoma, especially subjects with advanced melanoma and has undergone failed anti-cancer treatments with PD-1.

### FIELD

The present disclosure relates to the technical field of biological medicine, and specifically to a recombinant oncolytic herpes simplex virus type II (oHSV2) and a method for the treatment of melanoma therewith.

### BACKGROUND

Melanoma refers to a class of malignant tumors derived from melanocytes in tissues such as epidermis and mucous membranes. According to statistics, more than 280,000 new cases and more than 60,000 death cases are estimated in the world each year. A main reason responsible for such a high mortality rate is metastasis of a large number of tumor cells to the lymphatic system and other major organs in the body. Subjects in the early stage of melanoma are mainly treated with surgery, and other subjects, whose tumor cells have metastasized highly or spread to the lymph nodes, have to rely on treatments such as immunotherapy, chemotherapy, and radiation therapy. During the cancer development, melanin is often accompanied by mutations of genes or over-activation of proteins related to mitogen-activated protein kinase (MAPK) pathway, which over enhances the proliferation and invasive ability of tumor cells. Melanoma is a malignant immune tumor, which is more prevalent in people with low body immunity or those suffering from immunodeficiency conditions.

According to Chinese Society of Clinical Oncology (CSCO) guidelines, drugs used in the treatment of advanced melanoma mainly include chemotherapy drugs such as dacarbazine, temozolomide, paclitaxel, carboplatin, etc., targeted drugs such as for BRAF-positive, and PD-1 monoclonal antibodies. However, chemotherapy has limited efficacy on advanced melanoma in its first-line or second-line treatment, in which the objective response rate (ORR) of the first-line treatment ranges from 3.7% to 15%, with a median overall survival (mOS) ranges from 5.6 to 12.5 months. For example, dacarbazine, a solution in a standard first-line treatment recommended by CSCO, has an ORR of only 3.7% in Chinese subjects with advanced melanoma in the first-line treatment. It was demonstrated in a domestic multicenter randomized controlled double-blind study that the combination of dacarbazine and endostatin (endo) was more effective than dacarbazine alone in improving survival time of the subjects, where the mOS for the former was 12.0 months, while for the latter was 8.0 months.

Programmed death receptor-1 (PD-1) is a class of apoptosis-inhibitory proteins located on membranes of T-cells, which renders the T-cells non-functional by binding to PD-L1 of tumor cells. PD-1 antibodies play a role in anti-cancer based on the PD-1 monoclonal antibodies' competitive bindings to PD-1, thus eliminating the tumors' control on T cells, thereby disrupting tumor molecules. Pembrolizumab, also known as “K-drug”, is the first approved PD-1 monoclonal antibody for the second-line treatment of melanoma in China as recommended by CSCO, and is one of the main drugs currently used in the treatment of melanoma as well.

According to statistics, Pembrolizumab is used for the second-line treatment of unresectable or metastatic melanoma, and its clinical trial in the Chinese population showed an ORR of 16.7% and a mOS of 12.1 months. This is lower than the clinical efficacy data with an ORR of 41% in the European and American population, suggesting that acral melanoma (AM) prevalent in China is more intractable than cutaneous melanoma (CM) prevalent in Europe and America, and that may be attributed to the differences between their tumor mutation burdens and structure variances.

In 2018, toripalimab was approved by the National Medical Products Administration for the treatment of unresectable or metastatic melanoma in patients with failed systemic therapy previously. The study data shows that its ORR was 17.3%, and the mOS reached 22.2 months.

The above clinical trial results of the two PD-1 monoclonal antibody showed that PD-1 monoclonal antibody drugs enhanced the efficiency of advanced melanoma treatment. However, although PD-1 monoclonal antibody significantly improved the efficiency of the first-line or after-first-line treatment of advanced melanoma, the patients had failed PD-1 monoclonal antibody treatment or had PD-1 resistant were up to more than 82%.

A retrospective study conducted by Beijing Cancer Hospital analyzed 69 patients who accepted salvage treatment with Chemotherapy combined with antiangiogenic drugs (CA) after failed PD-1 monoclonal antibody (MAB) therapy, and found that among the 69 subjects, ORR of subjects treated with the first-line or second-line PD-1mab therapy in the initial stage was 11.59%, while ORR of subjects who continued the salvage therapy (albumin paclitaxel+endo/temozolomide+Apatinib) after failed PD-1mab therapy was only 5.8%.

The above results indicate that at present, although several drugs achieve certain therapeutic effects on treating melanoma, there is still a lack of effective treatment methods for advanced melanoma after standard first-line and second-line treatment failures. Accordingly, there is an urgent need to develop new melanoma therapeutic agents to meet clinical needs.

### SUMMARY

Regarding the deficiencies of the related art, in view of the limited therapeutic effects of currently approved drugs for the treatment of melanoma, an object of the present disclosure is to provide an antitumor drug with improved treatment efficacy, which is assessed by indicators such as “Objective Response Rate (ORR)”, “Disease Control Rate (DCR)”, “Median Overall Survival (mOS)”, and the like, and with fewer side effects for patients suffering from melanoma.

Oncolytic viruses refer to a new class of drug species, which are modified with genetic engineering techniques and have oncolytic activity, for the treatment of cancer. The oncolytic viruses, which may be natural or genetically edited, can specifically replicate in tumor cells and exert anti-tumor effects, and one of them, recombinant oncolytic herpes simplex virus, is an oncolytic viruses with development potential for cancer immunotherapy. Currently, there are four types of oncolytic viral therapies available on the worldwide market, among which adenoviruses, herpesviruses, reovirus, and cowpox virus are mainly applied, accounting for 31%, 24%, 20% and 12.5% respectively.

T-Vec, the first FDA-approved oncolytic virus drug, is a genetically modified herpes simplex virus type 1 (HSV-1) that replicates in tumor cells and expresses immune-activating proteins, for example, granulocyte-macrophage colony stimulating factor (GM-CSF). Injecting T-Vec directly into melanoma lesions leads to lysis of tumor cells, resulting in disruption of tumor cells, thereby releasing tumor-derived antigens and GM-CSF, to accelerate the anti-tumor immune response. However, T-Vec, as an oncolytic virus drug, still needs further improvements in the treatment of patients with advanced melanoma or PD-1 resistant melanoma.

Surprisingly, the inventors found that the recombinant oncolytic herpes simplex virus type II (oHSV2) shows an excellent therapeutic effect in the treatment of melanoma, especially for subjects with advanced melanoma or with failed PD-1 anti-cancer treatment and/or with PD-1 resistance. For the purpose of the present disclosure, the oHSV2, including a stable genomic sequence of a recombinant oncolytic herpes simplex virus type II as described in patent No. CN102146418B, refers to the modified herpes simplex type II virus obtained according to the method therein. As used herein, the term “oHSV2” refers to a recombinant oncolytic herpes simplex virus type II as disclosed in patent No. CN102146418B and Zhao Q, Zhang W, Ning Z, Zhuang X, Lu H, Liang J, Li J, Zhang Y, Dong Y, Zhang Y, Zhang S, Liu S, Liu B. A novel oncolytic herpes simplex virus type 2 has potent anti-tumor activity. PLOS One. 2014 Mar. 26; 9 (3): e93103. doi: 10.1371/journal.pone.0093103. PMID: 24671154; PMCID: PMC3966855. Meanwhile, the oHSV2, as an oncolytic virus capable of killing tumor cells, should be interpreted as an antitumor drug in the particular context of the present disclosure. The entire disclosure of patent No. CN102146418B is incorporated herein by reference.

To realize the above object, the present disclosure provides technical solutions as follows. In a first aspect, the present disclosure, in embodiments, provides an antitumor drug for melanoma, including recombinant oncolytic herpes simplex virus type II (oHSV2) as an active ingredient, which is named as H2d3d4-hGF, with a proposed taxonomic designation of Herpes Simplex Virus Type 2, and is deposited in depository authority of China General Microbiological Culture Collection Center located in Institute of Microbiology, Chinese Academy of Sciences, Building No. 3, Yard No. 1, West Beichen Road, Chaoyang District, Beijing, China, on Feb. 3, 2010, with an accession number of CGMCC No. 3600, where the oHSV2 is obtained by knocking out genes ICP34.5 and ICP47 of a wild herpes simplex virus type II strain HG52 and inserting a human granulocyte-macrophage colony-stimulating factor (hGM-CSF) cassette at the position of the knocked out gene ICP34.5.

In some embodiments, the antitumor drug is an oHSV2 injection.

In a second aspect, the present disclosure provides a method for treating a subject with melanoma, including: administering the subject a therapeutically effective amount of an antitumor drug as described in the first aspect embodiments of the present disclosure.

In some embodiments, the subject with melanoma is in a clinical stage of terminal phases III-IVMIa or IVMIb-IVMIc.

In some embodiments, the subject with melanoma is resistant to treatment with at least one anti-PD-1 monoclonal antibody.

In some embodiments, the subject with melanoma has experienced a failed PD-1 monoclonal antibody treatment.

In some embodiments, the subject with melanoma is in the clinical stage of terminal phases III-IVMIa and has experienced the failed PD-1 monoclonal antibody treatment and/or is resistant to treatment with at least one anti-PD-1 monoclonal antibody.

In some embodiments, the antitumor drug is in form of an injection.

In some embodiments, the oHSV2 in the antitumor drug is formulated in a pharmaceutically acceptable solution.

In some embodiments, the method further includes: administering the antitumor drug by intratumor injection.

In some embodiments, the antitumor drug is administered by a direct subcutaneous injection or an ultrasound-guided intratumor injection.

In some embodiments, the antitumor drug is administered once every 2 weeks, with an administration period of ≥6 months.

In some embodiments, the oHSV2 in the antitumor drug is administered with a dose from 10⁶ to 10⁸ CCID₅₀/ml.

In some embodiments, the oHSV2 in the antitumor drug is administered with a dose of 10⁶ CCID₅₀/ml, 10⁷ CCID₅₀/ml or 10⁸ CCID₅₀/ml with a single administration volume from 1 to 8 ml.

In some embodiments, the oHSV2 in the antitumor drug is administered with a dose lower than 8×10⁷ CCID₅₀.

In some embodiments, the antitumor drug is administered a dose or multiple doses.

In some embodiments, the antitumor drug may be used in a combination with other antitumor drugs, supportive (assistant) antitumor drugs and/or drug excipients.

The present disclosure provides an antitumor drug for melanoma, i.e. the oHSV2 injection. Compared to the related art, the antitumor drug provided by the present disclosure has at least the following beneficial effects.

i) The oHSV2 and its injection provided in the present disclosure is obtained by subjecting wild herpes simplex virus type II to modifications of knocking out neurotoxin and immunosuppressive genes and inserting immune-enhancing factor genes into its viral genome, with molecular cloning, DNA homologous recombination, and other techniques. The knock-out of neurotoxin genes enables the oHSV2 to selectively replicate in tumor cells with impaired PKR signaling pathway and expand to infect the surrounding tumor cells, rather than replicate in normal cells, which results in significantly decreased virulence and reduced drug side effects. The knock-out of the immunosuppressive genes facilitates the activation of anti-tumor immune responses. These two modifications enhance the oncolytic activity of the virus. In addition, insertion of hGM-CSF cassette induces differentiation, proliferation and maturation of tumors and their surrounding dendritic cell (DC) precursors, as well as enhances antigen presenting of DCs to activate immune killer cells in vivo, which contributes to inducing local and systemic anti-tumor immune responses.

ii) The oHSV2 and its injection provided in the present disclosure used for the treatment of melanoma show significantly improved ORR, DCR and mOS compared to the related art, achieving more beneficial therapeutic effects and reduced side effects caused by treatment in the meantime.

### BRIEF DESCRIPTION OF THE DRAWINGS

FIG. 1 is a schematic diagram showing changing trends of target lesions in the treatment with an oHSV2 injection in subjects with melanoma (n=37), compared to respective baselines, according to an embodiment of the present disclosure.

FIG. 2 shows efficacy analysis of the oHSV2 injection in the treatment on a subgroup of subjects with melanoma (n=22) during a dose expansion phase (phase Ib), according to an embodiment of the present disclosure.

FIG. 3 shows a preliminary survival analysis for the treatment of melanoma with an oHSV2 injection, according to an embodiment of the present disclosure.

### DETAILED DESCRIPTION

In order to make the object, technical solution and advantages of the present disclosure more clearly understood, the present disclosure is further described in detail below with reference to embodiments. It should be understood that the specific embodiments described herein are only intended to explain the present disclosure but not to limit the present disclosure.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Unless defined otherwise, all scientific and technical terms used herein have the same meaning as is commonly understood by one skilled in the art to which this invention belongs. All patents and publications referred to herein are incorporated by reference in their entirety.

### Terms

The term “oHSV2 injection” refers to oHSV2 in the form of injection. The term “injection” refers to a sterile solution containing drug and for injecting administration into the body, which includes an emulsion and suspension, as well as sterile powders or a concentrated solution containing drug and for preparation into a solution or suspension before use. The injection works rapidly and reliably and is free from influences of pH, enzymes, food, etc. as well as the first-pass effect, enabling systemic or local effects, and is therefore suitable for patients who are unfit for or cannot perform oral administration of drugs. In the present disclosure, the terms “injection” and “injection preparation” are equivalent in definition.

The terms “Complete Response (CR)”, “Partial Response (PR)”, “Stable Disease (SD)”, “Progressive Disease (PD)” refers to four assessment levels in terms of therapeutic efficacy on tumors (solid tumors only). Specifically, the term “CR” refers to all target lesions have disappeared, no new lesion appears, and a tumor markers are normal, with all of these maintaining for at least 4 weeks; “PR” refers to the sum of the largest diameters of target lesions has been reduced by ≥30%, and maintained for at least 4 weeks; “SD” refers to the sum of the largest diameters of target lesions is reduced but not reaching the criteria of PR, or increased but not reaching the criteria of PD; and “PD” refers to the sum of the largest diameters of target lesions has been increased by at least ≥20%, or new lesions appear.

The terms “Objective Response Rate (ORR)” and “Disease Control Rate (DCR)” refer to associated indicators for assessing the therapeutic efficacy on tumors (solid tumors only). Generally, “ORR” refers to the proportion of subjects whose tumor volumes have reduced by at least 30% and maintained for 4 weeks, that is to say, the sum of the proportions of subjects in CR and PR. The higher the ORR, the more subjects under the treatment having tumor reduced. “DCR” refers to the proportion of subjects that achieve CR+PR and SD under the treatment, i.e., the proportion of subjects who do not experience PD.

The term “Median Overall Survival (mOS)”, also known as half-survival in medicine, refers to the survival time corresponding to a cumulative survival rate of 50%, representing only 50% of the individuals being survival at this point. The mOS is an indicator for assessing the therapeutic efficacy of cancer subject in terms of survival, and is generally used to determine the prognosis of subjects with malignant tumors, where the longer the mOS, the longer overall survival time of subjects with malignant tumors. The mOS may also be used to determine the therapeutic efficacy of a new program. If subjects undergoing a certain treatment program is found to have a prolonged mOS or a significantly improved mOS than that of current standard treatment programs, generally such a new treatment program would be recommended or applied to clinical treatment, so as to bring certain benefits to the subjects such as increasing the survival rate of the subjects and improving the life quality of the subjects.

In embodiments of the present disclosure, clinical stages regarding melanoma, such as phases III-IVMIa, IVMIb-IVMIc, etc., are determined with reference to the American Joint Committee on Cancer (AJCC) Melanoma Staging Criteria (Eighth Edition).

In embodiments of the present disclosure, the wording “OH2-I-ST-01” refers to a clinical trial number of the oHSV2 injection for the treatment of melanoma as determined by inventors.

In embodiments of the present disclosure, the term “preferred” is only used for describing a more effective embodiment or example, and should not constitute a limitation on the protection scope of the present disclosure.

In embodiments of the present disclosure, the technical features described with an open manner both include a technical solution consisting of the enumerated features, and a technical solution including the enumerated features.

In embodiments of the present disclosure, when numerical intervals are involved, unless otherwise specified, endpoints of the numerical interval are included.

In embodiments of the present disclosure, an antitumor drug comprising oHSV2 may in addition comprise at least one pharmaceutically acceptable excipient/solution, e.g. carrier or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers.

Research on oHSV2 Injection and Anti-Tumor Mechanism Thereof

The recombinant oncolytic herpes simplex virus type II (oHSV2) injection provided in the present disclosure is obtained by subjecting a wild herpes simplex virus type II to modifications of knocking out neurotoxin and immunosuppressive genes and inserting immune-enhancing factor genes into the viral genome, with molecular cloning, DNA homologous recombination, and other techniques. The knock-out of neurotoxin genes enables the oHSV2 to selectively replicate in tumor cells with impaired PKR signaling pathway and expand to infect the surrounding tumor cells, rather than replicate in normal cells, which results in significantly decreased virulence and reduced drug side effects. The knock-out of the immunosuppressive genes facilitates the activation of anti-tumor immune responses. These two modifications enhance the oncolytic activity of the virus. In addition, insertion of hGM-CSF cassette induces differentiation, proliferation and maturation of tumors and their surrounding dendritic cell (DC) precursors, as well as enhances antigen presenting of DC to activate immune killer cells in vivo, which contributes to inducing local and systemic anti-tumor immune responses. It also ensures the oncolytic activity of the oHSV2 and increases its druggability along with immune activation.

The oHSV2 injection provided in an embodiment of the present disclosure includes a recombinant oncolytic herpes simplex virus type II, which is named as H2d3d4-hGF, with a proposed taxonomic designation of Herpes Simplex Virus Type 2, and is deposited in depository authority of China General Microbiological Culture Collection Center located in Institute of Microbiology, Chinese Academy of Sciences, Building No. 3, Yard No. 1, West Beichen Road, Chaoyang District, Beijing, China, on Feb. 3, 2010, with an accession number of CGMCC No. 3600.

The oHSV2 provided in an embodiment of the present disclosure has been verified for its therapeutic effects on tumors through several animal experiments before. According to Chinese patent No. CN102146418B, it was verified to the oncolytic effect and valid time of the oHSV2 on melanoma cells of Balb/c mice. The results showed that the oHSV2 had significant tumor-suppression effects on mouse melanoma, as the tumor size of the treated mice was significantly reduced. However, due to the significant individual differences between human and mice, the significant therapeutic effects of oHSV2 proved on animal (mouse) melanoma cannot be extended to good therapeutic effects on human melanoma, which still needs further verifications.

### Specific Example

In a specific example, there is provided a method for treating a melanoma tumor in a subject comprising administering the subject a therapeutically effective amount of a recombinant oncolytic herpes simplex type II virus, wherein the subject with melanoma is in the clinical stage III-IVMIa, and wherein the subject is resistant to treatment with at least one anti-PD-1 monoclonal antibody, and wherein the virus is formulated in a pharmaceutically acceptable solution, and wherein the virus strength is 10⁷ CCID₅₀ viral particles per 1 milliliter of pharmaceutically acceptable solution, and wherein the virus is administered every other week, and wherein said biweekly administration consists of a single or multiple direct or ultrasound-guided intratumoral injections, and wherein total viral particles administered per said biweekly injection do not exceed 8*10⁷ CCID₅₀ viral particles, and wherein the patient does not exhibit more than grade III or higher grade, treatment-related toxicities as defined by RECIST criteria within the first 4 administrations of treatment.

Clinical Assessment for Treatment of Melanoma with oHSV2 Injection

Examples of the present disclosure provide clinical use of oHSV2 and its injection in treatment of melanoma. Subjects with melanoma were administered with effective amounts of oHSV2 multiple times thereby maximizing the efficacy of the oHSV2 and avoiding or reducing side effects resulting from the administration. Specific examples are described below.

### Example 1. Clinical Trial Design

### 1.1 Phase Ia Clinical Trial

The primary objective of Phase Ia clinical trial was to explore the maximum tolerated dose (MTD) and dose limited toxicity (DLT) of oHSV2 injection, thereby evaluating the tolerability of the injection in the human body and providing a recommend safe dosage range for the Phase II clinical study of the virus injection; as well as to evaluate the biological distributions and effects of single and multiple intratumoral administration of the oHSV2 injection in the human body. The secondary objective was to preliminarily evaluate the anti-tumor efficacy of oHSV2 injection.

### 1.1.1 Main Enrollment Criteria

In the following criteria, items a-f were the enrollment criteria and g-j were exclusion criteria.

a. Subjects, who were definitively diagnosed with inoperable malignant melanoma at phase III or IV by pathology and/or cytology, were selected.

b. Subjects, whose cancer lacked conventionally effective treatment methods, or who had failed treatment or recurrence after treated with conventional methods, were selected.

c. Male or female subjects, who were aged from 18 to 75 years, of general physical condition score ECOG of 0 to 1 and expected survival for 3 months or more, were selected.

d. Subjects, who had been gone through for more than 4 weeks since finishing their prior antitumor therapy including endocrine, chemo/radiotherapy and targeted therapy, or for more than 6 weeks since finishing nitrosoureas and mitomycin chemotherapy, and had recovered to grade 1 from adverse effects of prior therapy, were selected.

e. Subjects, who had been gone through for 4 weeks since undergoing major surgery, were selected.

f. Subjects, who had at least one measurable lesion that was determined by CT or MRI according to Response Evaluation Criteria in Solid Tumors (RECIST, version 1.1) and was suitable for intratumoral injection, were selected, in which the term “measurable lesion” was defined as having a longest diameter ≥10 mm and a scan thickness of no more than 5.0 mm, and for lymph node lesion, defined as having a short diameter ≥15 mm.

g. Subjects, who were diagnosed with serious internal diseases including severe heart disease, cerebrovascular disease, uncontrolled diabetes mellitus, uncontrolled hypertension, serious infections, active peptic ulcers, and abnormalities of immune function such as autoimmune diseases, including but not limited to rheumatoid arthritis, lupus erythematosus, and sjogren's syndrome, etc., were excluded.

h. Subjects, who had uncontrolled primary or metastatic brain tumors, were excluded;

i. Subjects, who had uncontrolled psychiatric or infectious diseases, were excluded;

j. Subjects, with lesions that cannot meet the volume requirements for intratumoral injection, were excluded.

### 1.1.2 Subject Grouping and Administration Times

### A. Single Dosing Trial

This study was an open dose-escalation trial, which evaluated three incremental oHSV2-injection dose levels by single dosing. There were three groups as set, corresponding to three dose levels of 10⁶, 10⁷, 10⁸ CCID₅₀/ml. The oHSV2 injections at respective dose levels were administered intratumorally with single dosing. Each subject in each dose group was administered a maximum of 1 to 8 ml of the corresponding concentration dose of oHSV2 injection.

### B. Multiple Dosing Trial

There were two groups as set, corresponding to dose levels of 10⁷ CCID₅₀/ml and 10⁸ CCID₅₀/ml. The subject was administered 3 times of intratumoral injections repeatedly with 2-week intervals (or 3-week intervals, for HSV-2 serum antibody-negative subjects after the first induction). Each subject in each dose group was administered a maximum of 1 to 8 ml of the corresponding concentration dose of oHSV2 injection each time.

### 1.2 Phase Ib Clinical Trials

The phase Ib clinical trials were performed to further evaluate: i) the safety and tolerability of oHSV2 injection administered for treating advanced solid melanoma; ii) the anti-tumor efficacy of oHSV2 injection in treating solid melanoma; iii) biological distributions of oHSV2 injection in subjects with advanced solid melanoma; and iv) the immune responsiveness to oHSV2 injection in subjects with advanced solid melanoma.

### 1.2.1 Target Population: Subjects Who had Experienced Failed PD-Imab Therapy for Melanoma.

### 1.2.2 Phase Ib Recommended Dose (RP2D) was Determined by the Results of the Phase Ia Clinical Trials.

### 1.3 Drug Delivery

The oHSV2 injection was intratumorally administered to the selected tumor lesions. An acceptable injection volume of the test drug to the target injectable lesion was related to the long diameter of the lesion. Specifically, the maximum injection volume was 1 ml for a lesion with a long diameter ≤1.5 cm; 2 ml for a lesion with a long diameter >1.5 cm and ≤2.5 cm; 4 ml for a lesion with a long diameter >2.5 cm and ≤5.0 cm; and 8 ml for a long diameter ≥5 cm. For a subject with more than one injectable lesion, the largest target lesion was selected 5 for the first time injection. If the largest lesion was insufficient to inject 8 ml of drug, the remaining dose should be injected to other lesions, where for the second injection, new and/or enlarged target lesion(s) was selected. The injected drug was made to be evenly distributed into the tumor tissue, avoiding the drug overflow from the injection hole as far as possible. The injection should be implemented with sterile operation strictly.

### 2. Enrolled Subjects

A total of 44 subjects were enrolled in the Example, and 37 of them were screened out and subjected to imaging evaluation, whose baseline characteristics were shown in Table 1 below.

TABLE 1

| | | | | | | | HSV-2 | |
|---|---|---|---|---|---|---|---|---|
| Subject | | | | Disease | Staging | ECOG | serum | Previous treatment |
| number | Age | Sex | Dose group | diagnosis | at entry | score | antibody | history |
| 003 | 38 | Male | 10⁶CCID₅₀/ml | Melanoma in | PHASE IV | SCORE | POSITIVE | 2017 May 10-2017 Aug. 12 |
| | | | (Phase Ia- | right foot (AM) | (M1a) | 1 | | Interferon alpha-2b (PD); |
| | | | single dosing, | | | | | 2017 Dec. 18-2018 Jan. 30 |
| | | | “Ia single” | | | | | Apatinib targeted |
| | | | hereafter for | | | | | therapy (PD) |
| | | | short) | | | | | |
| 004 | 68 | Female | 10⁶CCID₅₀/ml | Melanoma in | PHASE IV | SCORE | POSITIVE | 2015 February DTIC + |
| | | | (Ia single) | right foot (AM) | (M1b) | 1 | | DDP chemotherapy (PD) |
| 005 | 47 | Female | 10⁶CCID₅₀/ml | Malignant | PHASE IV | SCORE | NEGATIVE | 2013 Aug. 4 Interferon |
| | | | (Ia single) | melanoma in | (M1c) | 1 | | alpha-2b, 8 months |
| | | | | left pelma | | | | (PD); reoperation; |
| | | | | (AM) | | | | 2014 Dec. 11 Interferon |
| | | | | | | | | adjuvant therapy; |
| | | | | | | | | 2014 Jun. 5 Dacarbazine + |
| | | | | | | | | cisplatin + endo for |
| | | | | | | | | 4 cycles (SD); |
| | | | | | | | | 2016 January Disease |
| | | | | | | | | progression; |
| | | | | | | | | 2016 Apr. 19-2016 June |
| | | | | | | | | SHR6390 tablet |
| | | | | | | | | targeted therapy (PD); |
| | | | | | | | | 2016 Aug. 16-2017 Jun. 5 |
| | | | | | | | | PD-1 monoclonal |
| | | | | | | | | antibody therapy |
| | | | | | | | | (Junmeng Biopharm) (PD); |
| | | | | | | | | 2017 Aug. 2-2018 Aug. 13 |
| | | | | | | | | Temozolomide + |
| | | | | | | | | Apatinib (targeted + |
| | | | | | | | | chemotherapy) (PD) |
| 006 | 55 | Female | 10⁷CCID₅₀/ml | Melanoma in | PHASE | SCORE | POSITIVE | 2015 June-2016 March |
| | | | (Ia single) | left foot (AM) | IIIC | 0 | | Interferon alpha-2b (9 |
| | | | | | | | | months) (PD); |
| | | | | | | | | 2016 April DTIC for 6 |
| | | | | | | | | cycles + radiotherapy to |
| | | | | | | | | right inguinal region (PD); |
| | | | | | | | | 2016 October |
| | | | | | | | | Temozolomide, 4 cycles (PD); |
| | | | | | | | | 2018 January-2018 April |
| | | | | | | | | Interferon therapy (PD) |
| 007 | 58 | Male | 10⁷CCID₅₀/ml | Acral | PHASE | SCORE | NEGATIVE | 2017 June-2017 December |
| | | | (Ia single) | Melanoma | IIIC | 1 | | Interferon alpha-1b |
| | | | | in the left | | | | (PD), re-excision; |
| | | | | heel (AM) | | | | 2018 Jan. 24-February 14 |
| | | | | | | | | High dose interferon |
| | | | | | | | | adjuvant therapy (PD); |
| | | | | | | | | 2018 Jun. 22 DTIC (PD); |
| | | | | | | | | 2018 Aug. 31-2018 Oct. 25 |
| | | | | | | | | Immunotherapy (JS001 240 |
| | | | | | | | | mg), 5 cycles (PD); |
| | | | | | | | | 2018 Dec. 29-2019 Jan. 7 |
| | | | | | | | | Albumin paclitaxel + |
| | | | | | | | | carboplatin |
| 008 | 62 | Male | 10⁷CCID₅₀/ml | Nodular | PHASE IV | SCORE | NEGATIVE | 2017 Apr. 6-2017 Jun. 5 |
| | | | (Ia single) | melanoma | (M1b) | 1 | | DTIC + DDP + Endo (PD); |
| | | | | in the right | | | | 2017 Jul. 19-2018 Nov. 8 |
| | | | | upper arm | | | | Clinical trial of PD-1 |
| | | | | | | | | antibody therapy, |
| | | | | | | | | unknown drug (PD) |
| 010 | 18 | Male | 10⁸CCID₅₀/ml | Blue nevus | PHASE IV | SCORE | NEGATIVE | 2016 November-2017 February |
| | | | (Ia single) | melanoma in | (M1a) | 0 | | DTIC + DDP + Endo + |
| | | | | right calf | | | | IL-2 + IFN; |
| | | | | | | | | 2017 June-2018 January |
| | | | | | | | | High dose interferon |
| | | | | | | | | therapy (PD); |
| | | | | | | | | 2018 Jun. 11-2018 Oct. 29 |
| | | | | | | | | Paclitaxel + Bevacizumab |
| | | | | | | | | (humanized anti-VEGF |
| | | | | | | | | monoclonal antibody) (PD) |
| 011 | 56 | Female | 10⁸CCID₅₀/ml | Superficial | PHASE IV | SCORE | NEGATIVE | 2017 Feb. 20-2017 Jun. 19 |
| | | | (Ia single) | spreading | (M1a) | 0 | | DTIC + DDP, 5 cycles (PD); |
| | | | | melanoma in | | | | 2018 Jan. 25-2018 Aug. 2 |
| | | | | the left calf | | | | Temozolomide + Endo (PD) |
| 013 | 54 | Female | 10⁸CCID⁵⁰/ml | Uterine | PHASE | SCORE | NEGATIVE | 2017 Apr. 19 |
| | | | (Ia single) | myoma/ | IIID | 0 | | Postoperative interleukin + |
| | | | | superficial | | | | interferon for 2 months; |
| | | | | spreading | | | | 2017 August Postoperative |
| | | | | malignant | | | | interleukin + interferon |
| | | | | melanoma, | | | | for 20 days, +endo for 14 |
| | | | | right foot | | | | days (PD); Reoperation; |
| | | | | melanoma | | | | 2018 Feb. 7 high dose |
| | | | | | | | | interferon treatment, (PD); |
| | | | | | | | | 1997 Excision with |
| | | | | | | | | postoperative chemotherapy + |
| | | | | | | | | radiotherapy; |
| | | | | | | | | 2018 Nov. 2-2019 Mar. 6 |
| | | | | | | | | Keytruda PD-1 MAB |
| | | | | | | | | therapy, 7 cycles (PD) |
| 014 | 63 | Male | 10⁷CCID₅₀/ml | Malignant | PHASE III | SCORE | NEGATIVE | 2017 Dec. 27-2018 September |
| | | | (Phase Ia, | melanoma in | | 0 | | Interferon therapy, 9 |
| | | | multiple | right toe (AM) | | | | months (PD); |
| | | | dosing, “Ia | | | | | 2018 December-2019 Mar. 23 |
| | | | multiple” | | | | | Dacarbazine + cisplatin, |
| | | | hereafter | | | | | 5 cycles (PD) |
| | | | for short) | | | | | |
| 015 | 72 | Male | 10⁷CCID₅₀/ml | Malignant | PHASE III | SCORE | NEGATIVE | 2018 July-2018 September |
| | | | (Ia multiple) | melanoma | | 0 | | Interferon therapy (PD) |
| | | | | of the left | | | | |
| | | | | pelma (AM) | | | | |
| 016 | 73 | Female | 10⁷CCID₅₀/ml | Metastatic | PHASE IV | SCORE | NEGATIVE | 2018 December 25-2019 Jan. 8 |
| | | | (Ia multiple) | melanoma of | (M1a) | 0 | | Temozolomide + Endo (PD); |
| | | | | lymph node in | | | | 2019 Mar. 13-2019 Jun. 5 |
| | | | | left inguinal | | | | HX008 PD-1 MAB therapy (PD) |
| 017 | 69 | Female | 10⁸CCID₅₀/ml | Melanoma in | PHASE III | SCORE | NEGATIVE | 2018 Apr. 28-2018 December |
| | | | (Ia multiple) | left foot (AM) | | 1 | | High dose interferon |
| | | | | | | | | adjuvant therapy (PD); |
| | | | | | | | | 1984 Excision, |
| | | | | | | | | followed by 2 cycles of |
| | | | | | | | | DTIC + DDP + VCR + BCNU |
| | | | | | | | | postoperatively; |
| | | | | | | | | excision on March 29, |
| | | | | | | | | 2018, 2 cycles of |
| | | | | | | | | chemotherapy DTIC + |
| | | | | | | | | DDP + VCR + BCNU |
| | | | | | | | | postoperatively (PD) |
| 018 | 52 | Female | 10⁸CCID₅₀/ml | Malignant | PHASE IV | SCORE | NEGATIVE | 2016 Nov. 26-2017 October |
| | | | (Ia multiple) | melanoma in | (M1b) | 1 | | High-dose interferon |
| | | | | the right | | | | therapy (PD); |
| | | | | foot (AM) | | | | 2019 Jan. 9 Vemurafenib |
| | | | | | | | | (SD, no significant |
| | | | | | | | | shrinkage, later |
| | | | | | | | | recommended in |
| | | | | | | | | combination with PD-1 |
| | | | | | | | | MAB therapy); |
| | | | | | | | | 2019 May 16-2019 Jul. 28 |
| | | | | | | | | Vemurafenib + |
| | | | | | | | | Toripalimab (PD); |
| | | | | | | | | 2019 July interferon |
| | | | | | | | | therapy with local |
| | | | | | | | | injection (PD) |
| 019 | 41 | Female | 10⁸CCID₅₀/ml | Malignant | PHASE IV | SCORE | POSITIVE | 2018 June-2018 September |
| | | | (Ia multiple) | melanoma in | (M1a) | 1 | | Adjuvant interferon |
| | | | | the right | | | | therapy, 3 months (PD); |
| | | | | lower limb | | | | 2019 Apr. 2 Albumin |
| | | | | (AM) | | | | paclitaxel + |
| | | | | | | | | carboplatin + endo (PD); |
| | | | | | | | | 2019 May 1-2019 Jun. 16 |
| | | | | | | | | Albumin paclitaxel + |
| | | | | | | | | Toripalimab, 2 cycles (PD); |
| | | | | | | | | 2019 Jul. 2-July 15 |
| | | | | | | | | Anlotinib + Toripalimab |
| 020 | 59 | Male | 10⁷CCID₅₀/ml | Nodular | PHASE IV | SCORE | NEGATIVE | 2018 Mar. 17 Interferon |
| | | | (Ib multiple) | melanoma in | (M1c) | 0 | | adjuvant therapy, 11 |
| | | | | right foot | | | | months |
| 021 | 66 | Male | 10⁷CCID₅₀/ml | Cutaneous | PHASE IV | SCORE | NEGATIVE | 2015 High dose |
| | | | (Ib multiple) | malignant | (M1b) | 1 | | interferon therapy |
| | | | | melanoma in | | | | (duration unknown); |
| | | | | the right | | | | reoperation; |
| | | | | forearm | | | | 2017 Nov. 30-2018 November |
| | | | | | | | | Interferon therapy (PD); |
| | | | | | | | | 2018 Nov. 20-2019 April |
| | | | | | | | | Keytruda |
| | | | | | | | | immunotherapy (PD); |
| | | | | | | | | 2019 May 1 Keytruda + |
| | | | | | | | | Axitinib (PD); |
| | | | | | | | | 2019 Nov. 28-2020 Apr. 22 |
| | | | | | | | | Paclitaxel + |
| | | | | | | | | carboplatin (PD) |
| 022 | 68 | Female | 10⁷CCID₅₀/ml | Nodular | PHASE III | SCORE | NEGATIVE | 2017 Mar. 27-2017 Jul. 21 |
| | | | (Ib multiple) | malignant | | 1 | | Interferon adjuvant |
| | | | | melanoma in | | | | therapy (PD); |
| | | | | the right | | | | 2017 Aug. 4 Lipid |
| | | | | pelma (AM) | | | | paclitaxel + Endo, 2 |
| | | | | | | | | cycles (PD); |
| | | | | | | | | 2017 Sep. 7-2017 Sep. 14 |
| | | | | | | | | Apatinib (PD); |
| | | | | | | | | 2017 Oct. 25-2019 Nov. 12 |
| | | | | | | | | Hengrui PD-1 antibody (PD) |
| 023 | 64 | Female | 10⁷CCID₅₀/ml | Malignant | PHASE | SCORE | NEGATIVE | 2019 May-2019 July |
| | | | (Ib multiple) | melanoma in | IIIB | 1 | | High dose interferon |
| | | | | the right | | | | therapy (PD) |
| | | | | foot (AM) | | | | |
| 024 | 69 | Female | 10⁷CCID₅₀/ml | Cutaneous | PHASE IV | SCORE | NEGATIVE | 2015 Aug. 12-2016 Aug |
| | | | (Ib multiple) | melanoma in | (M1b) | 1 | | High dose interferon |
| | | | | the right palm | | | | therapy, 11 months (PD) |
| 025 | 60 | Female | 10⁷CCID₅₀/ml | Melanoma in | PHASE | SCORE | NEGATIVE | 2019 Jul. 26-2020 Jul. 10 |
| | | | (Ib multiple) | the left pelma | IIIC | 1 | | TQB2450 (targeted |
| | | | | (AM) | | | | PD-L1) + Anlotinib, |
| | | | | | | | | 16 cycle (PD), for |
| | | | | | | | | targeted therapy |
| 026 | 49 | Male | 10⁷CCID₅₀/ml | Melanoma in | PHASE IV | SCORE | NEGATIVE | 2019 Oct. 25-2020 Jul. 12 |
| | | | (Ib multiple) | the left | (M1a) | 1 | | Immunotherapy |
| | | | | foot (AM) | | | | (Toripalimab) (PD) |
| 027 | 57 | Male | 10⁷CCID₅₀/ml | Scalp | PHASE IV | SCORE | NEGATIVE | 2016 Sep. 1-2017 September |
| | | | (Ib multiple) | melanoma | (M1a) | 1 | | Interferon alpha-2b |
| | | | | | | | | treatment for 1 year (PD); |
| | | | | | | | | 2019 Nov. 12-2020 Mar. |
| | | | | | | | | 30sintilimab |
| | | | | | | | | immunotherapy (PD) |
| 028 | 71 | Female | 10⁷CCID₅₀/ml | Melanoma in | PHASE | SCORE | NEGATIVE | 2018 Nov. 1-2019 November |
| | | | (Ib multiple) | left toe (AM) | IIIC | 1 | | High dose interferon |
| | | | | | | | | adjuvant therapy for |
| | | | | | | | | 11 months (PD) |
| 029 | 60 | Female | 10⁷CCID₅₀/ml | Cutaneous | PHASE | SCORE | NEGATIVE | 2019 Jun. 28 High dose |
| | | | (Ib multiple | nodular | IIIC | 0 | | interferon therapy for |
| | | | times) | melanoma in | | | | 3 weeks (stopped after |
| | | | | the right thigh | | | | side effects); |
| | | | | | | | | 2020 May 9-2020 Sep. 10 |
| | | | | | | | | Adjuvant therapy with |
| | | | | | | | | Toripalimab after the |
| | | | | | | | | second postoperative |
| | | | | | | | | operation (PD) |
| 030 | 67 | Male | 10⁷CCID₅₀/ml | Malignant | PHASE IV | SCORE | NEGATIVE | 2015 Dec. 29-2016 December |
| | | | (Ib multiple) | melanoma in | (M1c) | 1 | | 11 months of high- |
| | | | | the right | | | | dose interferon |
| | | | | pelma (AM) | | | | therapy; reoperation; |
| | | | | | | | | 2018 June-2019 Nov. 28 |
| | | | | | | | | Interferon adjuvant |
| | | | | | | | | therapy for 11 months (PD); |
| | | | | | | | | 2015 October-2015 Dec. 25 |
| | | | | | | | | Dacarbazine + |
| | | | | | | | | interleukin + interferon, |
| | | | | | | | | chemotherapy, 3 cycles (PD); |
| | | | | | | | | 2019 Dec. 31-2020 Dec. 1 |
| | | | | | | | | HX008 immunotherapy (PD) |
| 031 | 58 | Male | 10⁷CCID₅₀/ml | Right | PHASE | SCORE | NEGATIVE | 2018 Jul. 20 DTIC |
| | | | (Ib multiple) | plantar | IIIB | 1 | | 2018 Aug. 17-2018 Oct. 24 |
| | | | | melanoma | | | | TP; |
| | | | | (AM) | | | | 2019 Jun. 11 Interferon |
| | | | | | | | | immunotherapy; |
| | | | | | | | | 2019 Sep. 19 Interferon |
| | | | | | | | | alpha-2b + |
| | | | | | | | | interleukin-2 (PD); |
| | | | | | | | | 2020 Jan. 17-2020 Jul. 28 |
| | | | | | | | | Chemotherapy combined |
| | | | | | | | | with immunotherapy |
| | | | | | | | | (Toripalimab); |
| | | | | | | | | 2020 Sep. 2 |
| | | | | | | | | Immunotherapy with |
| | | | | | | | | Toripalimab |
| 032 | 75 | Male | 10⁷CCID₅₀/ml | Melanoma in | PHASE IV | SCORE | NEGATIVE | Direct enrollment |
| | | | (Ib multiple) | right foot (AM) | (M1b) | 0 | | after operation due to |
| | | | | | | | | lack of conventional |
| | | | | | | | | treatment at the time |
| 033 | 70 | Male | 10⁷CCID₅₀/ml | Nevus | PHASE IV | SCORE | NEGATIVE | 2016 Oct. 31-2017 September |
| | | | (Ib multiple) | melanoma in | (M1b) | 0 | | High-dose adjuvant |
| | | | | right toe (AM) | | | | interferon therapy, 11 months |
| 034 | 51 | Female | 10⁷CCID₅₀/ml | Left plantar | PHASE IV | SCORE | POSITIVE | 2016 March Interferon |
| | | | (Ib multiple) | melanoma (AM) | (M1a) | 0 | | alpha-2b adjuvant |
| | | | | | | | | therapy (PD); |
| | | | | | | | | 2020 August-2021 February |
| | | | | | | | | Toripalimab, 8 cycles (PD); |
| | | | | | | | | 2021 Mar. 8-2021 Mar. 30 |
| | | | | | | | | Camrelizumab (PD-1) + |
| | | | | | | | | Endo + albumin- |
| | | | | | | | | paclitaxel + carboplatin |
| 035 | 49 | Female | 10⁷CCID₅₀/ml | Melanoma in | PHASE IV | SCORE | NEGATIVE | 2017 May 25-2018 March |
| | | | (Ib multiple) | right foot (AM) | (M1b) | 0 | | High-dose interferon |
| | | | | | | | | therapy, 12 months (PD) |
| | | | | | | | | 2020 Jul. 28-2021 Mar. 14 |
| | | | | | | | | PD-1 MAB therapy (PD); |
| | | | | | | | | 2020 October-2021 Apr. 2 |
| | | | | | | | | Localized interferon |
| | | | | | | | | injection in lesions (PD); |
| | | | | | | | | 2021 Apr. 9 Tocilizumab |
| | | | | | | | | therapy (recombinant |
| | | | | | | | | humanized anti-human |
| | | | | | | | | interleukin 6 (IL-6) |
| | | | | | | | | receptor monoclonal |
| | | | | | | | | antibody) (PD) |
| 036 | 60 | Female | 10⁷CCID₅₀/ml | Lentigo acral | PHASE IV | SCORE | NEGATIVE | 2021 Jun. 29 Performed |
| | | | (Ib multiple) | melanoma in | (M1a) | 0 | | surgery, enrolled in |
| | | | | the left foot | | | | oHSV2 clinical trial, |
| | | | | | | | | enrolled directly after |
| | | | | | | | | surgery due to lack of |
| | | | | | | | | conventional |
| | | | | | | | | treatment at the time |
| 037 | 52 | Male | 10⁷CCID₅₀/ml | Melanoma in | PHASE IV | SCORE | NEGATIVE | 2020 September-2021 Jan. 18 |
| | | | (Ib multiple) | the left pelma | (M1b) | 1 | | Toripalimab (PD); |
| | | | | (AM) | | | | 2021 Apr. 7-2021 May 20 |
| | | | | | | | | KD6001 (CTLA-4 antibody) |
| 038 | 70 | Female | 10⁷CCID₅₀/ml | Superficial | PHASE IV | SCORE | NEGATIVE | 2017 November-2018 Apr. 28 |
| | | | (Ib multiple) | spreading | (M1a) | 0 | | DTIC, 6 cycles + |
| | | | | melanoma in | | | | interleukin-II biotherapy (PD); |
| | | | | the left foot | | | | 2018 Nov. 7-2018 Dec. 4 |
| | | | | | | | | Docetaxel + |
| | | | | | | | | Carboplatin + Endo (PD) |
| | | | | | | | | 2019 Jan. 16-2021 Jun. 18 |
| | | | | | | | | Pembrolizumab (PD) |
| 039 | 58 | Male | 10⁷CCID₅₀/ml | Melanoma of | PHASE IV | SCORE | NEGATIVE | 2020 Sep. 5-2021 Mar. 26 |
| | | | (Ib multiple) | left pelma | (M1c) | 0 | | High-dose interferon |
| | | | | (AM) | | | | therapy, 6 months (PD) |
| | | | | | | | | 2021 Apr. 26-2021 Jul. 20 |
| | | | | | | | | Temozolomide + |
| | | | | | | | | Camrelizumab + Apatinib (PD) |
| 040 | 60 | Female | 10⁷CCID₅₀/ml | Nasal | PHASE III | SCORE | NEGATIVE | 2018 Jul. 11-2019 Jul. 8 |
| | | | (Ib multiple) | melanoma | | 1 | | anti-PD-1 monoclonal |
| | | | | (mucosal | | | | antibody injection; |
| | | | | melanoma) | | | | 2020 Sep. 4-2022 Apr. 22 |
| | | | | | | | | HX008 + LP002 |
| | | | | | | | | Clinical trial, assessed |
| | | | | | | | | as SD at cycles 2nd |
| | | | | | | | | and 4th, PR at cycle |
| | | | | | | | | 8th PR maintained |
| | | | | | | | | thereafter, and iUPD |
| | | | | | | | | at cycle 28th during |
| | | | | | | | | the treatment period |
| 041 | 47 | Female | 10⁷CCID₅₀/ml | unknown | NA | SCORE | NEGATIVE | 2022 Apr. 18 Enrolled |
| | | | (Ib multiple) | primary | | 0 | | after puncture biopsy |
| | | | | melanoma | | | | of left supraclavicular |
| | | | | | | | | lymph node metastasis |

### 3. Results of Clinical Trial

### 3.1 Safety

In this Example, treatment emergent adverse events are shown as Table 2.

TABLE 2
Treatment Emergent Adverse Event (TRAE)

| | | Number of | | |
|---|---|---|---|---|
| | | subjects | | |
| | | involved | | Times of |
| Preferred term | Grade | in cases (%) | | cases |
| Total | 1-2 | 28 | (75.7) | 86 |
| Fever | 1-2 | 23 | (62.2) | 44 |
| Loss of skin pigmentation | 1 | 6 | (16.2) | 6 |
| Limb pain | 1-2 | 3 | (8.1) | 4 |
| Skin rash | 1 | 3 | (8.1) | 3 |
| Nausea | 1 | 3 | (8.1) | 3 |
| Elevated gamma-glutamyltransferase | 1 | 2 | (5.4) | 4 |
| Skin swelling | 1 | 2 | (5.4) | 2 |
| Decreased appetite | 1 | 1 | (2.7) | 1 |
| Hyperglycemia | 1 | 1 | (2.7) | 1 |
| Skin infections | 2 | 1 | (2.7) | 1 |
| Elevated creatine phosphokinase | 1 | 1 | (2.7) | 1 |
| Elevated aspartate aminotransferase | 1 | 1 | (2.7) | 1 |
| Decreased white blood cell count | 1 | 1 | (2.7) | 1 |
| Elevated conjugated bilirubin | 1 | 1 | (2.7) | 3 |
| Elevated blood bilirubin | 1 | 1 | (2.7) | 4 |
| Elevated platelet count | 1 | 1 | (2.7) | 1 |
| Decreased neutrophil count | 1 | 1 | (2.7) | 1 |
| Dry pharynx | 1 | 1 | (2.7) | 1 |
| Skin peeling | 1 | 1 | (2.7) | 1 |
| Hair loss | 1 | 1 | (2.7) | 1 |
| Peripheral edema | 1 | 1 | (2.7) | 1 |
| fatigue | 1 | 1 | (2.7) | 1 |

As shown in Table 2, adverse reactions occurred in 28 subjects of 37 subjects for 86 times, most of the cases were evaluated as grade 1 for the severity of adverse reactions, and a small number of them were as grade 2. The common adverse reactions were fever, loss of skin pigmentation, limb pain and the like, without serious adverse events. The results above showed that oHSV2 injection is of a high safety.

### 3.2 Efficacy

3.2.1 Efficacy Analysis on OH2-I-ST-01 at Phase Ia/Ib (n=37)

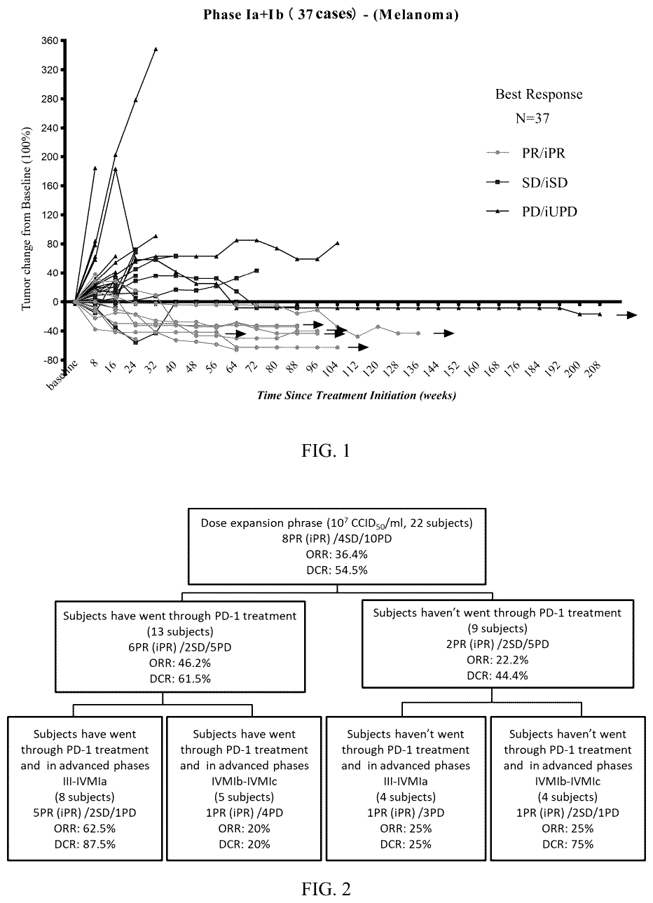

FIG. 1 shows changing trends of target lesions in subjects with melanoma (n=37) in the treatment with oHSV2 injection. As can be seen from the figure, the treatment results of the 37 subjects received the treatment were as follows: 8 subjects were assessed as in PR, 13 subjects were assessed as in SD, and 16 subjects were assessed as in PD, with ORR of 21.6%, and DCR of 56.8%, demonstrating well efficacy achieved by the oHSV2 injection in the treatment of melanoma.

### 3.2.2 Efficacy Analysis on OH2-I-ST-01 at Phase Ib Subgroup (N=22)

Phase Ib subgroup (N=22), referred to the subjects therein who received oHSV2 injection during dose expansion phase (Phase Ib) in the treatment of melanoma. Among the 22 subjects, 13 of them had received PD-1 MAB therapy, and 9 of them had not. Among the 13 subjects who had received PD-1 MAB therapy, 8 of them were subjects with advanced melanoma at phase III-IVMIa, and 5 subjects with advanced melanoma at phase IVMIb-IVMIc. Among the 9 subjects who had not received PD-1 MAB therapy, 4 of them were subjects with advanced melanoma at phase III-IVMIa, 4 were subjects with advanced melanoma at phase IVMIb-IVMIc, and 1 was a subject with unknown primary lesions and unknown tumor phase.

FIG. 2 shows efficacy analysis of the oHSV2 injection according to Examples of the present disclosure in the treatment on a subgroup of subjects with melanoma (n=22) during a dose expansion phase. As shown in the figure, treatment results of the subjects advanced with melanoma at II-IVMIa phase that had received PD-1 MAB therapy were as follows: 5 subjects were assessed as in PR, 2 subjects were assessed as in SD, and 1 subject was assessed as in PD, with ORR of 62.5%, and DCR of 87.5%, demonstrating greatly improved efficacy.

3.2.3 Comparison of Efficacy Between oHSV2 Injection and Common PD-1 Mab Drugs

The comparison of efficacy data between oHSV2 injection and common PD-1 monoclonal antibody drugs in the treatment of melanoma subjects is shown in Table 3 below.

TABLE 3

| | | Number of | | | mOS |
|---|---|---|---|---|---|
| Drug Name | Population | Population | ORR | DCR | (month) |
| Pembrolizumab | Failed in first-line treatment | 103 | 16.7% | 38.2% | 12.1 |
| | | | | | (not reached) |
| Toripalimab | Failed in first-line treatment | 128 | 17.3% | 57.5% | 22.2 |
| Pucotenlimab | Failed in conventional | 119 | 20.17% | 43.7% | 16.59 |
| | therapy | | | | |
| oHSV2 | Failed in second-line | 22 | 36.4% | 54.5% | 26.1 |
| injection | treatment | | | | (not reached) |
| | | 37 | 21.6% | 56.8% | 25.6 |
| | | | | | (not reached) |

Several common PD-1 monoclonal antibodies for melanoma treatment and their therapeutic effects are listed in Table 3. Among them, pembrolizumab was the first PD-1 monoclonal antibody approved in China for treating melanoma, used for the second-line treatment of unresectable or metastatic melanoma, with an ORR of 16.7% and a mOS of 12.1 months in its clinical trial conducted for the Chinese population; toripalimab was also approved by the National Medical Products Administration for the treatment of unresectable or metastatic melanoma subjects who had failed in previous systemic therapy, according to the study data, its ORR was 17.3% and mOS was 22.2 months; and pucotenlimab, approved as PD-1 monoclonal antibody drug in 2022 for subjects with locally advanced or metastatic melanoma who have failed in conventional treatment, such as chemotherapy, targeted therapy, interferon, IL-2, etc., has an ORR of 20.17% and a mOS of 16.59 months, according to the study data. By contrast, the oHSV2 injection provided by Examples of the present disclosure showed a significant improvement in both the ORR and mOS compared with the three former PD-1 monoclonal antibody drugs.

3.2.4 Survival Data in the Treatment of Melanoma with oHSV2 Injection

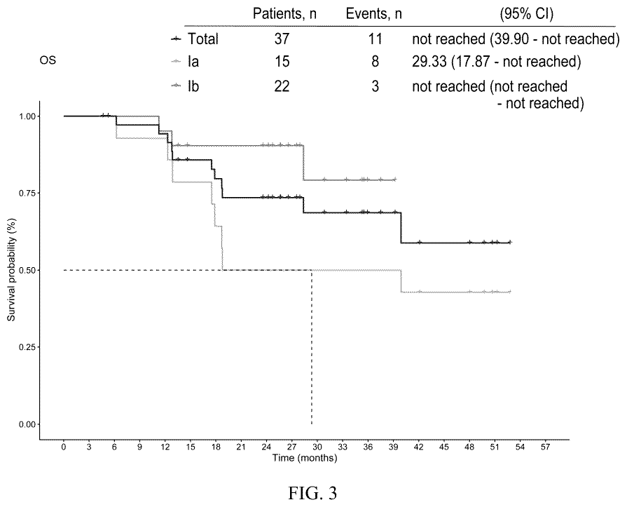

FIG. 3 shows a preliminary survival analysis for the treatment of melanoma by the oHSV2 injection according to Examples of the present disclosure. By the end of June 2023, the mOS of 37 subjects enrolled in the study has not reached yet, with a median follow-up time of 25.6 months; and the mOS of 22 subjects in the clinical group at phase Ib has not reached neither; and in the continued follow-up, the mOS of 15 subjects at dose escalation phase Ia was 29.3 months.

In summary, the oHSV2 injection provided by Examples of the present disclosure for the treatment of subjects with melanoma, showed significantly improved ORR, DCR and mOS, thereby achieving more effective therapy and reduced side effects caused by the treatment compared to the prior drugs.

What is described above is only a better specific embodiment of the present disclosure, the scope of protection of the present disclosure is not limited thereto. Any changes or substitutions that can be readily thought of by any person skilled in the art within the technical scope disclosed in the present disclosure shall be covered by the scope of protection of the present disclosure.

What is claimed is:

1. A method for treating a subject with melanoma, comprising:
administering the subject a therapeutically effective amount of an antitumor drug,
wherein the antitumor drug comprises recombinant oncolytic herpes simplex virus type II (oHSV2) with a deposit number of CGMCC No. 3600 as an active ingredient.

2. The method according to claim 1, wherein the subject with melanoma is in a clinical stage of terminal phases III-IVMIa or IVMIb-IVMIc.

3. The method according to claim 2, wherein the subject with melanoma is resistant to treatment with at least one anti-PD-1 monoclonal antibody.

4. The method according to claim 1, wherein the subject with melanoma is in a clinical stage of terminal phases III-IVMIa and is resistant to treatment with at least one anti-PD-1 monoclonal antibody.

5. The method according to claim 2, wherein the subject with melanoma has experienced a failed PD-1 monoclonal antibody treatment.

6. The method according to claim 1, wherein the antitumor drug is in form of an injection, the method further comprises:
administering the antitumor drug by intratumor injection.

7. The method according to claim 1, wherein the antitumor drug is administered by a direct subcutaneous injection or an ultrasound-guided intratumor injection.

8. The method according to claim 6, wherein the oHSV2 in the antitumor drug is formulated in a pharmaceutically acceptable solution.

9. The method according to claim 1, wherein the antitumor drug is administered once every 2 weeks.

10. The method according to claim 1, wherein the antitumor drug is administered with an administration period of ≥6 months.

11. The method according to claim 1, wherein the oHSV2 in the antitumor drug is administered with a dose from 10⁶ to 10⁸ CCID₅₀/ml.

12. The method according to claim 11, wherein the oHSV2 in the antitumor drug is administered with a dose of 10⁶ CCID₅₀/ml, 10⁷ CCID₅₀/ml or 10⁸ CCID₅₀/ml with a single administration volume from 1 to 8 ml.

13. The method according to claim 1, wherein the oHSV2 in the antitumor drug is administered with a dose lower than 8×10⁷ CCID₅₀.

14. The method according to claim 11, wherein the antitumor drug is administered a single dose or multiple doses.

15. The method according to claim 1, wherein the antitumor drug is used in a combination with supportive antitumor drugs or drug excipients.

16. The method according to claim 1, wherein the oHSV2 is obtained by knocking out genes ICP34.5 and ICP47 in a wild herpes simplex virus type II strain HG52 and inserting a human granulocyte-macrophage colony-stimulating factor (hGM-CSF) cassette at the position of the knocked out gene ICP34.5.
